(19) United States
(12) Patent Application Publication
(10) Pub. No.: US 2006/0216256 A1
(43) Pub. Date: Sep. 28, 2006

(54) Foaming oral care compositions of baking soda and vinegar

(76) Inventors: Martin S. Giniger, New York, NY (US); Matthew S. Spaid, New York, NY (US); Brian Olson, New York, NY (US)

Correspondence Address: HENSLEY KIM & EDGINGTON, LLC, 1660 LINCOLN STREET, SUITE 3050, DENVER, CO, 80264, US

(21) Appl. No.: 11/355,502

(22) Filed: Feb. 15, 2006

Related U.S. Application Data
(60) Provisional application No. 60/653,421, filed on Feb. 15, 2005.
(60) Provisional application No. 60/734,549, filed on Nov. 7, 2005.
(60) Provisional application No. 60/739,634, filed on Nov. 26, 2005.

(51) Int. Cl. A61K 8/00

(52) U.S. Cl. 424/70.1

(57) ABSTRACT

A foaming oral care composition of two parts; namely, a vinegar part, and a baking soda part. Also included are alternative additional components such as lemon oil, orange oil, xanthan gum or an oxidizing whitener such as hydrogen peroxide or sodium chlorite. Methods for making and using such an oral care composition are included as well.

### BACKGROUND

This invention relates generally to oral care compositions such as whiteners for teeth, and more particularly to foaming oral care compositions.

In the state of the art of oral care compositions and the delivery thereof to the site of use in the oral cavity, many means and methods have been utilized and yet numerous issues remain. For an effective ingredient of an oral care composition to have a therapeutic effect, whether for oral cleaning, treatment or tooth whitening, the effective ingredient must reach and be maintained in effective contact with the oral care feature long enough to provide its intended effect. Thus, dispersion and penetration into and between the surfaces of various oral features such as the odd shapes of the nooks and crannies of adjacent teeth is a continual issue. So too then is the dwell or contact time necessary or at least preferred for having the effective ingredient or ingredients of an oral care composition maintained in contact with or otherwise disposed adjacent the surface of the oral feature being cared for. Such issues arise in various oral cleaning, treatment and/or tooth whitening situations.

In tooth cleaning and/or treatment, effective ingredients such as fluoride or an anti-gingival agent, e.g., triclosan, must reach the areas between teeth or between a tooth and gums and/or reach the nooks and crannies on/of teeth to provide their benefits to those oral features.

Similar activities are necessary in tooth whitening as well. In considering tooth whitening generally, it may first be noted that a tooth is comprised of an inner dentin layer and an outer hard enamel layer that is the protective layer of the tooth. The enamel layer of a tooth is naturally an opaque white or slightly off-white color. It is this enamel layer that can become stained or discolored. The enamel layer of a tooth is composed of hydroxyapatite mineral crystals that create a somewhat porous surface. It is believed that this porous nature of the enamel layer is what allows staining agents and discoloring substances to permeate the enamel and discolor the tooth.

Many substances that a person confronts or comes in contact with on a daily basis can “stain” or reduce the “whiteness” of one's teeth. In particular, the foods, tobacco products and fluids such as tea and coffee that one consumes tend to stain one's teeth. These products or substances tend to accumulate on the enamel layer of the tooth and form a pellicle film on the teeth. These staining and discoloring substances can then permeate the enamel layer. This problem occurs gradually over many years, but imparts a noticeable discoloration of the enamel of one's teeth.

There are available to dentists and consumers many different oral compositions for home and professional in-office use which contain 1-45% by weight concentrations of a peroxygen compound such as hydrogen peroxide and when applied on the teeth may effect whitening of stains. These compositions all require different amounts of time to achieve a desired tooth bleaching effect. These times range from 90 to 120 minutes for a dentist applied, light-activated bleaching system to two weeks or more of over night exposure for tray-delivered whitening products. Currently, even the top selling brands of dentist applied, light activated chair-side tooth whitening systems require a minimum of three (3) twenty-minute applications and an overall minimum of ninety (90) minutes or more to complete when all manufacturers' instructions have been followed.

Among the chemical strategies available for removing or destroying tooth stains, the most effective compositions contain an oxidizing agent, usually a peroxygen compound such as hydrogen peroxide, in order to attack the chromogen molecules in such a way as to render them colorless, water-soluble, or both. In one of the most popular approaches to whitening a patient's teeth, a dental professional will construct a custom-made tooth-bleaching tray for the patient from an impression made of the patient's dentition and prescribe the use of an oxidizing gel to be dispensed into the tooth-bleaching tray and worn intermittently over a period of time ranging from about 2 weeks to about 6 months, depending upon the severity of tooth staining. These oxidizing compositions, usually packaged in small plastic syringes, are dispensed directly by the patient, into the custom-made tooth-bleaching tray, held in place in the mouth for contact times of greater than about 60 minutes, and sometimes as long as 8 to 12 hours. The slow rate of bleaching is in large part the consequence of the very nature of formulations that are developed to maintain stability of the oxidizing composition.

Alternatively, there are oxidizing compositions (generally those with relatively high concentrations of oxidizers) which are applied directly to the tooth surface of a patient in a dental office setting under the supervision of a dentist or dental hygienist. Theoretically, such tooth whitening strategies have the advantage of yielding faster results and better overall patient satisfaction.

Oral compositions for whitening teeth have also been available containing peracetic acid dissolved or suspended in a vehicle. The peracetic acid may have been generated within a dentifrice vehicle by combining water, acetylsalicylic acid and a water soluble alkali metal percarbonate.

Formulations for oxygen liberating compositions for the whitening of teeth have also used either anhydrous and/or hydrated pastes or gels. Hydrated examples include an aqueous oral gel composition comprising about 0.5 to about 10% by weight urea peroxide and 0.01 to 2% by weight of a fluoride providing compound, and/or a water containing a hydrogen peroxide-Pluronic thickened oral gel composition.

Other examples include a toothpaste containing a combination of calcium peroxide and sodium perborate oxidizing agents, dicalcium phosphate, calcium carbonate and magnesium carbonate cleaning agents, sorbitol humectant, cornstarch and cellulose gum thickening agents, and an anionic detergent, and/or oral compositions containing peroxyacids and alkyl diperoxy acids having alkylene groups containing 5-11 carbon atoms for removing stains from teeth.

Yet another conventional example includes administering a light-activated gel under the supervision of a dentist using a protocol of a usual three (3) twenty minute applications. Patients frequently become uncomfortable, agitated and/or bored during such a procedure that typically lasts 1.5 to 2 hours when all set-up and precautionary methods have been included. Also, because of the length of exposure to both the gel and the light, teeth and oral tissues can become irritated or experience a transient hypersensitivity reaction. Thus, any improvement that can result in decreased time, increased patient comfort and increase in bleaching efficiency is desirable.

More specific background information on activating bleaching agents with light energy includes the following. Scientists have identified many kinds of UV photoactivators, which are capable of working in nature to reduce the color of chromophoric stains. These include: transition metal complexes, keto acids, riboflavin, pteridines, algal pigments, cyanocobalamine, thiamin, biotin and aromatic ketones. The pathways by which photo beaching can theoretically occur on tooth surfaces are of two types. First, if the absorption spectrum of the colored chromagen overlaps with the spectrum of incoming radiation, the substrate may undergo photoreaction directly—e.g., the notion of fading color with light. Secondly, and likely a more powerful means for effecting color changes, UV energy may be absorbed by photo activators that then react with tooth surface chromagens, resulting in an “indirect” photobleaching.

Indirect photobleaching may be mediated by transient species (free radicals) that are rapidly consumed by subsequent reactions. For these mechanisms, the rate of reaction is determined by the quantity and type of chromagen, activator, free radicals and incoming UV radiation. Surface gradients involving any of these factors will lead to altered rates of photobleaching at the enamel/bleaching agent interface.

In nature, the major photochemical intermediate free radicals include singlet oxygen, 1O2; superoxide O2-, hydroperoxide HO2° and various other peroxy radicals, RO2. These have been described in more than one hundred patents for the purpose of bleaching teeth. Singlet oxygen free radicals (the most common type of free radical liberated from hydrogen peroxide in the presence of light, heat or most activators), 1O2, are formed primarily through energy transfer from the excited triplet states of dioxygen, 3O2 (as seen in the case of hydrogen peroxide), and wavelengths in the UV-A (315400 μm) and UV-B (280-315 nm) have been shown to be most effective in their formation. Quantum yields (the fraction or percentage of absorbed photons which give rise to products) range from 1 to 3% and generally decrease with increasing wavelength. Because the high concentrations of hydrogen peroxide or similar compounds are present in tooth bleaching preparations, its decay into water and 1O2 is dominated by this pathway when UV light/activator systems are used in professional tooth bleaching formulas.

The exact mechanism of how these singlet oxygen free radicals come to be formed still remains unclear. Some researchers have suggested that 1O2 is formed by direct electron transfer from the excited triplet states to O2. However, reduction of O2 by radicals or radical ions produced by intramolecular electron transfer reactions, H-atom abstractions and/or homolytic bond cleavages, is equally, if not more plausible. However, it is known that transition metal complexes having one-electron reduction potentials falling between the O2/O2- and O2-/H2O2 couples can rapidly catalyze 1O2 free radical formation.

A commercial application has been made of oxidation from the photo-fenton reaction in which reduced metals such as Fe(II) react with H2O2 and UV light to produce a single OH-radical. This may be because hydroxyl moieties may be generated with less UV activation energy reduction in a chromophoric tooth stain in a given period of time or for a given level of UV energy (the high quantum yield for this reaction is 98%).

These extant methods are not quickly nor highly effective and indeed typically need prolonged periods for any minimum effective bleaching effects. These time-consuming methods thus suggest that any whitening system that can reduce the time factor is desirable.

### SUMMARY

The present invention is directed generally to a combination of baking soda and vinegar to provide a foaming oral care composition such as a tooth whitener. In particular, disclosed here are two component oral care compositions such as a whitener or cleaner which includes a baking soda component and a vinegar component. These two components may be kept separate until use at which point they may be mixed to create a foaming composition which may be applied to the oral care feature such as a tooth. The baking soda and vinegar create a foaming composition which may be an effervescent type of foam which may include small conveniently sized bubbles and/or may provide a half life of about 10 minutes.

In another view, the present invention relates to two-component foaming oral care compositions. More particularly, herein described are two-component foamable compositions having a first component including at least one vinegar compound; and a second component including a baking soda or a sodium bicarbonate compound. A surfactant may also be included in either one or both of the first and or second components. The resultant composition may act as a tooth cleaner and/or as a tooth whitening agent. A peroxide may be added to the vinegar component to provide a tooth whitening composition. Other additives may be included as described below.

The detailed description set forth herein below is intended as a description of a variety of exemplar oral care products including baking soda and vinegar compositions provided in accordance with one or more aspects of the present invention and is not intended to represent the only forms which may be prepared or utilized. The description may set forth features and/or operations for preparing and using the oral care compositions according hereto. It is to be understood, however, that the same or equivalent functions and ingredients incorporated in such compositions may be accomplished by different embodiments that are nevertheless also intended to be and are encompassed within the spirit and scope of the present invention.

### DETAILED DESCRIPTION OF THE DRAWINGS

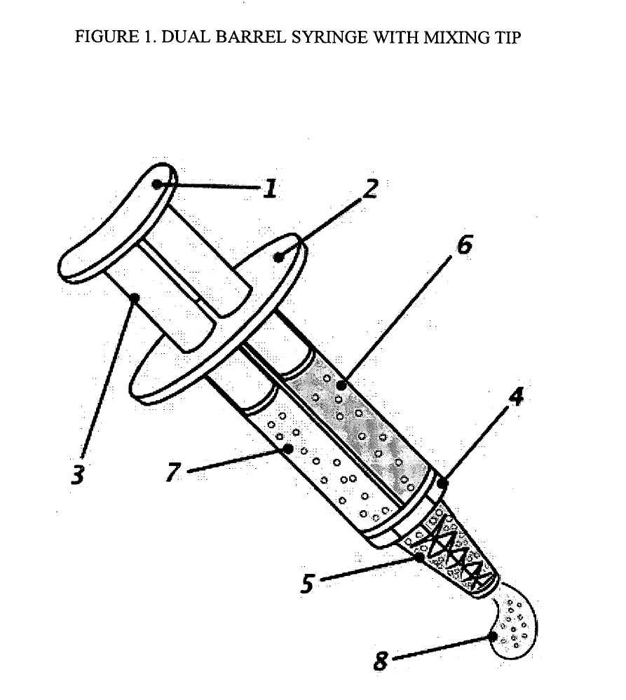

FIG. 1 is a dual-chamber syringe useful for dispensing compositions hereof;

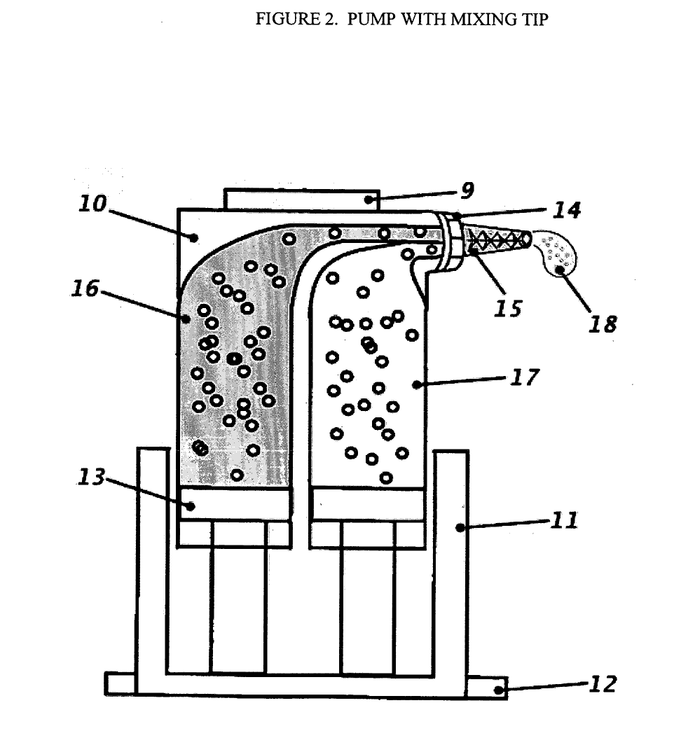

FIG. 2 is a dual-chamber dispenser useful for dispensing compositions hereof; and,

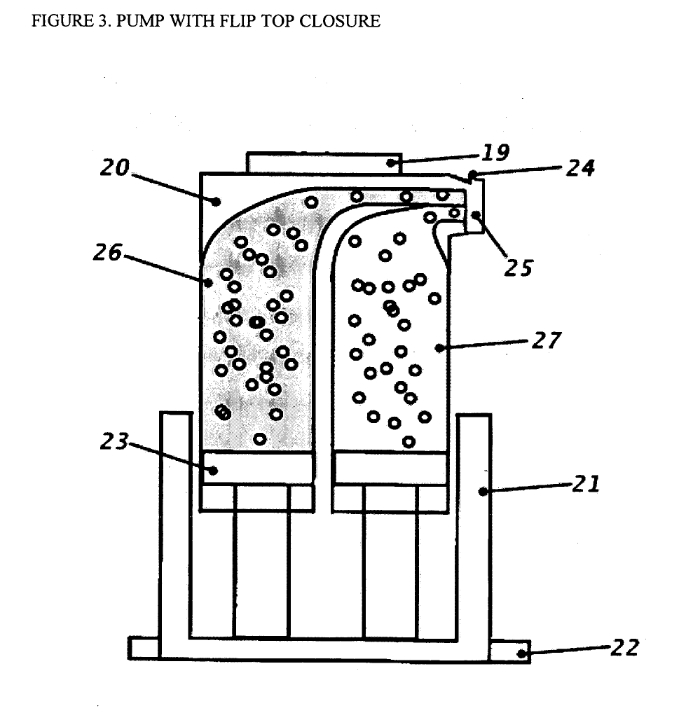

FIG. 3 is another dual-chamber dispenser useful for dispensing compositions hereof.

### DETAILED DESCRIPTION

The detailed description set forth herein is intended as a description of several of the presently exemplified oral care compositions provided hereby and is not intended to represent the only forms in which such compositions may be prepared or utilized. The description sets forth the features and the steps for preparing and using the oral care compositions hereof. It is to be understood, however, that the same or equivalent functions and ingredients incorporated in the oral care compositions may be accomplished by different embodiments that are also intended to be encompassed within the spirit and scope hereof.

As used herein, foaming and more particularly effervescent foaming refers to a growing foam, generally of substantially small, or otherwise substantially consistently-sized, orally-acceptable bubbles. An effervescent foaming composition hereof generally includes compositions which evolve gas by means of a chemical reaction between an acid and a base (e.g., an alkaline source), or a vinegar and a bicarbonate such as sodium bicarbonate which takes place upon exposure of the vinegar and the bicarbonate to each other. Thus, the vinegar and bicarbonate are maintained separate (even if but merely in separate phases in a common compartment) until use when the separate components are brought into contact with each other to create a foaming oral care composition.

Each of the vinegar and/or the bicarbonate may also be disposed in or otherwise be brought into contact with water or other fluids. Liquid forms of the respective components may provide stability and decrease viscosity, lower viscosities contributing to enhanced dispersion and penetration. Moreover, the effervescence and/or foaming is here generally used to also enhance dispersion as well as increase dwell time in the oral cavity of the active ingredient(s) of the effervescent foaming oral care composition. Controlled and/or consistent bubble formation in an effervescent foam such as those herein can thus also contribute to better oral care, cleaning and in some embodiments, whitening of teeth. Gas eruption in the effervescent foaming hereof is orally-acceptable, and thus is typically slow and gentle, however, more explosive foaming may also be usable herein/herewith so long as such remains orally acceptable (e.g., not causing undesirable damage, as to any of the oral tissues or surfaces). Foaming agents hereof are thus those generally of a chemical nature, including chemical agents that aid in foam generation, as well as aiding in sustained foaming, generating a gas that produces foaming, or in assisting a composition to become self-effervescent, or an agent that produces similar results. Again, though generally of a chemical reaction nature herein, a foaming agent hereof may alternatively include a mechanical foaming initiator or contributor as well.

In a first group of embodiments, the compositions hereof may include two components; a first one of which containing a vinegar, and the second one of which containing a bicarbonate, typically a sodium bicarbonate. Either or both of these components may then also include a surfactant. Such a two-component composition may then provide an effervescent foam when the first and second components are brought into contact with each other. Indeed, no agitation or external gas need be added in many such embodiments. A surfactant such as poloxamer 188 and/or poloxamer 407 may be used to control bubble size; e.g., smaller bubbles. Two other foam stabilizers may be Xanthan gum, particularly a clear xanthan gum and/or EDTA either or both of which typically being disposed in the vinegar component. Another alternative foam stabilizer may be propylene glycol alginate or a sodium alginate. These may provide for bubble size maintenance and/or for longer lasting bubbles/foams. Such a composition, when formed into a foam can be used to clean oral tissues or surfaces; in one example, such may form a denture cleanser. In many embodiments, these first and second components are liquids which enhance stability and reduce viscosity.

A foamable composition such as those hereof is thus manageable during use, is capable of delivering high concentration of oral care or cleaning compounds without confinement problems. The foamed composition can also seek to fill crevices and gaps (nooks and crannies) between teeth or other tissue surfaces. Foam also has the ability to drain into and seep or penetrate into gaps where gaps are not apparent, thus offering effective whitening action not only to the front surface of a tooth, but surfaces between teeth as well. Contrarily, previous viscous compositions could not flow between the surfaces, and thickeners inhibited the availability of oral care compounds from reaching the intended intrinsic surfaces.

Aqueous solutions and gels without foaming action or surfactants tend to hold drops of water together as they try to become spherical, which is the reason why water tends to flow in tendrils rather than in sheets. At the atomic level, surface tension is seen as a macro effect of unbalanced electrical forces at the surface of the liquid, the same electrical forces that hold the molecules of the liquid together and keep it from evaporating. Within the body of a typical aqueous solution or gel, there are lots of molecules in all directions, and electrical forces are balanced on the average. A molecule is pulled equally in all directions by neighboring molecules, resulting in no net force on it. On the surface, however, there are only a scant few air molecules to counteract the pull of the liquid below. The result is an inward-directed force pulling on a molecule near the surface, ultimately forming an electrical armor that envelops the liquid like an ultra thin skin. Thus, it is easy to see that non-foaming oral care gels tend to hold on to their active ingredients. Water molecules near the surface of gels intensify and magnify the surface tension, resulting in a so called “skin” effect by aligning to form a type of “elastic electrical force field” that seals and shields the surface even more. The shape of the water molecule leaves one of its ends with a slightly positive charge and the other end with slightly negative. Because of this electrical asymmetry (known as polarization), the negative and positive ends of adjacent molecules attract one another, creating a weak but significant physical bond. Energy is then required to stretch and break the bonds, and spreading out the liquid would mean that some of the bonds have to be broken. In this manner, most of the active oral care molecules in a typical oral gel have difficulty escaping from the surface tension bondage to perform their intended function.

Foams in general have lower surface tension than their liquid solution counterparts prior to foaming and can therefore be spread as thin as one molecule thick sheets, while at the same time increasing their surface areas. Thus, it is clear that foamable compositions hereof have advantages over gels and solutions. Without being bound to any particular theory, it is surmised that active oral care ingredients can be captured inside the bubbles of foams. As the bubbles collapse, the active agents may be released to perform oral care actions. Also, the foamed bubbles, through the forces of capillary action and lowered surface tension, are also wetting agents, an oral care solution can penetrate through smaller openings by weakening the electrically charged “skin of surface tension” typically found in aqueous solutions and gels. The rapid penetration into the tightest spots is also aided by the distribution of bubble sizes and shapes. Thus, foams can cause deeper penetration of active peroxide molecules. Additionally, foaming agents can increase the adhesion of a liquid to a solid surface by allowing it to spread over a greater surface area.

It is further surmised that aside from the ability to lower surface tension, foams can also act to propel the active oral care molecules toward the teeth and also act as a molecular “Lint Roller” to grab stains or other undesirable features and lift them off of tooth surfaces.

Other foaming agents can include reaction products of any effervescent compound with an acid. The effervescent compound, i.e., basic compound, also known as an alkaline source, is, for example, a pharmaceutically acceptable alkali metal carbonate or bicarbonate, such as sodium bicarbonate, potassium bicarbonate, sodium carbonate or potassium carbonate. The typical exemplar used in the below-presented examples is sodium bicarbonate. The amount used can range, for example, from about 1% to about 10% by weight, inter alia. The amount used depends on the volume of foam required, the desired pH and the desired diffusional or proper osmotic activity of the foam to be formed.

Generally, the ratio of acid and basic compound, i.e., vinegar to sodium bicarbonate may range from, for example, about 1:0.5 to about 1:25, though a variety of relationships may be used. An example amount may be of from, for example, about 0.5% to about 5% by weight of the composition, though here also other amounts may be used.

The first and/or second components may typically be liquids and will often include a carrier. Water (H2O) is a common carrier. In many embodiments, these first and second components are both liquids which may enhance stability and reduce viscosity.

In another group of embodiments, compositions hereof may include at least one whitening agent such as a peroxide compound or a sodium salt of chlorous acid such as sodium chlorate or sodium chorite or chlorine dioxide to create a tooth whitening composition (though it may remain an oral care cleanser as well, see above). The peroxide may be hydrogen peroxide or may be one or more of other peroxides such as those for example, which may include metal ion free peroxide compounds. Thus, examples of suitable metal ion free peroxide compounds include hydrogen peroxide and organic peroxides including urea peroxide (carbamide peroxide), salts of peroxides formed from the alkali and alkaline earth metals, glyceryl peroxide, benzoyl peroxide and the like. A few of the examples of the peroxide to be included in the first and/or second component are hydrogen peroxide or a mixture of hydrogen peroxide and carbamide peroxide. Others include calcium peroxide and/or mixtures thereof with either or both hydrogen and/or carbamide peroxide. The total peroxide present in the foamable oral care composition, may for example, range from about 1% by weight to about 45% by weight of the composition, further for another example from about 5% by weight to about 35% by weight of the composition or for a further example from about 10% to about 20% by weight of the composition.

When hydrogen peroxide is used, it is usually provided as an approximate 50% aqueous solution. When used alone, the amount of the hydrogen peroxide aqueous solution in the first component may range from, for example, about 2% to about 90% (1% to 45% in the absence of water), or as a further example, the amount may range from about 10% to about 60% (5% to 30% in the absence of water). On the other hand, when carbamide peroxide is used, it may often be used in combination with hydrogen peroxide, though it can be used alone. When used in combination, the carbamide peroxide may then generally be present in an amount from, for example, just slightly more than about 0% by weight to about 40% by weight, and, for a further example, in an amount from about 3% to about 35% by weight. Hydrogen peroxide, generally provided as a 50% aqueous solution, may be present in an amount of from, for example, about 1% (w/w) to about 30% (w/w) (about 0.5% to about 15% in the absence of water); or for a further example, in an amount of about 5% (w/w) to about 30% (w/w) (about 2.5% to about 15% in the absence of water).

A peroxide such as those described above may be present in one or the other or both components of the composition. Typically however, the peroxide will be in the vinegar (acid) containing component, the first component described above. Thus, the peroxide may be included in the first component in a range of from just above about 0% (w/w) to about 50% (w/w).

It has been understood that peroxides such as hydrogen peroxide, carbamide peroxide, and others such as salts of peroxides formed from the alkali and alkaline earth metals, readily attack and oxidize organic molecules. Known generally also is that such organic molecules are often involved in the stains in discolored teeth. Thus, there is some general understanding that peroxides are effective to oxidize and remove organic stains in/on teeth. Thus, when one or more peroxides are used, the oral care composition(s) may be tooth whitening compositions. Moreover, it is also understood that in certain circumstances, such oxidizing activity can be accelerated by the addition of heat, light and/or chemicals, particularly chemicals that can raise the pH of the peroxide environment. A dissertation of the possible mechanisms is presented in U.S. Pat. No. 6,116,900, “Binary Energizer and Peroxide Delivery System for Dental Bleaching” which is incorporated herein by reference.

In addition to the alternative addition of heat, light and/or chemicals, the amount of whitening obtained during tooth bleaching with peroxide compositions has previously generally been dependent upon (1) the length of time the teeth are in contact with the whitening agent; (2) the number and/or length of periods (e.g., hours and/or days) the treatment is carried out; (3) the susceptibility of the teeth to the bleaching agent; and (4) the concentration of active peroxide, as noted above. For maximum whitening, a long treatment time with a highly concentrated bleaching composition had generally been recommended.

Bleaching activity of a peroxide compound has generally been dictated by the availability of active peroxides. When peroxide is present in solution, active peroxides are readily available. However, a solution, by its nature, is not easily contained, and/or not amenable for sustained action when applied to a patient's teeth, again because it is difficult to confine it to any desired location. Thus, a less concentrated peroxide solution requiring longer contact time to be effective is not practical, while a more concentrated solution of peroxide, though more efficient in bleaching, still does not solve the confinement problem, and any concentrated solution coming into contact with soft tissue inside a patient's mouth can potentially cause tissue damage. Therefore, to maintain effective bleaching without potential tissue damage, various gelling agents, thickeners and adhesion promoters have been used to form pastes, gels, and similar forms to achieve prolonged contact effects. Unfortunately, such additives used to achieve prolonged contact also help to decrease the bleaching activity of peroxides by inhibiting the availability of active peroxides. For example, bleaching gels usually contain thickeners derived from polymers of acrylic acid (carbomer), pyrrolidone analog thickeners, or others, all of which leading to diminishing the whitening capacity of peroxides through ionic and covalent interactions within the gel, and thus acting against the desired effect of tooth whitening.

As described above, a foamable composition such as those hereof can, on the other hand, solve the problems encountered by gels and the like. A foamed composition is manageable during use, is capable of delivering a high concentration of peroxide (or other oral care or cleaning compounds) without confinement problems, while at the same time will not seriously inhibit the availability of active peroxides. The foamed composition can also seek to fill crevices and gaps between enamel rods. Foam also has the ability to drain into and seep into gaps where gaps (nooks and crannies) are not apparent, thus offering effective whitening action not only to the front surface of a tooth, but surfaces in-between teeth as well. A viscous composition that cannot flow between the enamel rods, and thickeners that inhibit the availability of active peroxides for tooth whitening, the present invention provides all the benefits not currently available to other whitening systems.

Since the whitening activity results when active peroxide comes into contact with the tooth, the foams generated, for example, are not too long lasting, but have a substantive body so that the amount of liquid formed, i.e., the collapsing of the foam bubbles to release active peroxide can be controlled and balanced. Therefore, the preferred compositions are not only foamable, but are also capable of producing longer lasting, collapsible foams.

Though not required, such foams may thus have half lives of, for example, from about 2 to 60 minutes, or for a further example, of at least about 5 to about 30 minutes, and in a preferred embodiment of about 10 or about 10.1 minutes. A foam having a half life of about 10 minutes means that about 50% of the bubbles would collapse to release the encapsulated whitening agent, or that the volume of the foam is reduced by about 50% in about 10 minutes after formation, and about 75% of all the bubbles are gone, or the volume of the foam is reduced by about 75% in about 20 minutes. The collapse time or half lives of the foam depends on a number of factors. For example, the greater the amount of bubbles formed, the longer the collapse time. Also, the lower the viscosity of the stabilizer, the shorter the collapse time. The collapse time also depends on the nature of the other additives to the composition, which have surface active properties e.g., surfactants or preservatives.

The longer lasting foams can, for example, be generated by including a foam stabilizer in the composition. The foam stabilizer is, for example, a hydrogel-like mixture of gum and water. For example, the viscosity of a solution thickened with the stabilizer remains substantially the same in a wide pH range and is relatively independent of ionic strength and the thickener can be, for example, xanthan gum, hydroxyethyl cellulose having about 1000-5000 cps, or and thereof. The advantage of xanthan gum is that compositions containing it can have a low viscosity at high shear rates, and are thus relatively easy to pump, spray or spread. Xanthan gum also exhibits a high viscosity at low shear rates, thus preventing the composition from dripping or flowing into places where it is not wanted. Thus, in addition to good stabilization of foams, it also gives it the substantive body desired. Xanthan gum is also widely used in the food industry and thus is safe for consumption.

Of particular note here is an alternative use of a clear xanthan gum, rather than a regular xanthan gum which has acids that can eat right through the foam. A clear xanthan gum hereof is one which has been hydrated for a period, as for example a period of about 24 hours, for provision of long term stability. Such a foam stabilizer can provide a desirable stiffening to the foam.

The stabilizer may be, for example, used in such concentrations that solutions or dispersions containing it in its pre-swelled form have viscosities as measured by Rotational Viscometer CSR-10a of within the range of, for example, less than about 50 cps, further for example, less than about 20 cps, still further for example, less than about 10 cps. The concentration of stabilizers can range, for example, from about 0.1 to about 10%, further for example, from about 0.5 to about 5%, still further for example, less than about 1% by weight of the composition. When xanthan gum is used, it may be, for example, present from about 0.5 to about 2% by weight of the composition, and in one implementation at about 0.9%. When hydroxyethyl cellulose is present, it is, for example, ranging from about 0.5 to about 2% by weight of the composition.

Of further note are two other of myriad possible additives; namely lemon oil, orange oil or orange extract. The lemon oil may rather be substituted by a citric acid. The combination of these four known foods (baking soda, vinegar, lemon and orange) which are also known as cleaners is thought to be novel and/or brings novel aspects to an oral composition, particularly a foaming whitener described herein.

The compositions and/or methods hereof are further described by the following examples:

### EXAMPLE 1

The ingredients for making an exemplary composition according hereto are set out in Table 1 below (percentages are approximates by weight of the respective components; carriers and/or respective acid and/or base materials can be added to Q.S.). They may be prepared as follows.

Component 1 (acidic) may be prepared by mixing and/or dissolving a hydrated/clear xanthan gum (preferably hydrated for about 24 hours) in water; 0.9% xanthan gum to 59.8% water. Added hereto may be dehydrated alcohol, 3.5% acetic acid, 0.35% citric acid (or lemon oil as described above), 0.1% EDTA (another foam stabilizer, particularly for long lasting effects), 0.25% sodium citrate as a buffer, 0.1% sodium benzoate, 10% vinegar and 20% of a 50% hydrogen peroxide solution.

Component 2 (basic) may be prepared by adding to a 17% water carrier, 5% dehydrated alcohol, 0.35% orange extract or orange oil may be added for flavor, as well as for its cleansing effect (note the orange and/or lemon may be added to either or both components of the two-component product hereof), 1.1% poloxamer 188 and 0.6% poloxamer 407. To this solution may be added 0.5% titanium dioxide, 1.2% sodium saccharin, 4% potassium nitrate (for sensitivity control), 0.1% cetyl pyridinium chloride (a preservative and/or antimicrobial), 6.5% sodium bicarbonate and enough potassium hydroxide to make this component basic, pH of about 8.0-9.0, and in one implementation preferably about 8.8.

Then, when desired to use the combination product, components 1 and 2 are mixed to create an effervescent foam which may be applied to the teeth, dentition or other oral feature. Three preferable features include the use of about 2-4 natural food products to beneficial use, these products being natural and effective; namely, baking soda, vinegar, orange and lemon. Second, a long-lasting, stable foam is created here which can help maximize the exposure and reach of the effective ingredients into and around the oral feature, as for example of a peroxide around a tooth in a whitening use. And, third, the use of the clear xanthan gum creates a good foam stabilizer without any use of carbomers which scavenge free radicals such as those used in peroxide (and other) whiteners.

In alternative instances hereof, oral care compositions hereof may thus also/alternatively include a surfactant which may be a part of or define a foaming agent. Suitable surfactants may be anionic, nonionic, amphoteric, zwitterionic, cationic, and/or mixtures thereof. Some of these many kinds of surfactants aid in foam formation and some do not. Some surfactants are useful purely for their foaming properties alone, some act only as emulsifiers or wetting agents without foaming, and some may even act to reduce foaming. Herein, the surfactants may include, for example, those that not only have foaming capabilities, but also those with the ability to act as wetting agents. Anionic surfactants include, but are not limited to water-soluble salts of alkyl sulfates having from 8 to 20 carbon atoms in the alkyl radical (e.g., sodium alkyl sulfate), water-soluble salts of sulfonated monoglycerides of fatty acids having from 8 to 20 carbon atoms and mixtures thereof. Examples of anionic surfactants include sodium lauryl sulfate, sodium coconut monoglyceride sulfonates, phospholipids, sarcosinates, such as sodium lauryl sarcosinate, taurates, sodium lauryl sulfoacetate, sodium lauroyl isethionate, sodium laureth carboxylate, and sodium dodecyl benzenesulfonate. Many of these anionic surfactants are disclosed in U.S. Pat. No. 3,959,458, the content of which is incorporated herein in its entirety by reference. Nonionic surfactants can include, but are not limited, to compounds comprising a hydrophilic and hydrophobic components (which may be produced by the condensation of alkylene oxide groups (hydrophilic in nature) with an organic hydrophobic compound which may be aliphatic or alkyl-aromatic in nature). Examples of suitable nonionic surfactants include low viscosity poloxamers (under trade name Pluronic), low viscosity hydroxyethyl cellulose, polysorbates, polyoxyethylene sorbitan esters (under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures thereof. Amphoteric surfactants can include, but are not limited to derivatives of aliphatic secondary and tertiary amines in which the aliphatic component can be a straight chain or branched and one of the aliphatic substituents contains from about 8 to about 18 carbon atoms and one contains an anionic water-solubilizing group, e.g., carboxylate, sulfonate, sulfate, phosphate, phosphonate, betaines, specifically cocamidopropyl betaine, and mixtures thereof. Many of these nonionic and amphoteric surfactants are disclosed in U.S. Pat. No. 4,051,234, the content of which is incorporated herein by reference in its entirety.

Actually, any asymmetrical molecule dissolved in water will make at least a weak surfactant. Such weak surfactants may normally not be an effective foaming agent, but its effectiveness can be improved if an alternatively available foaming dispenser is used. Asymmetrical molecules as used herein may include those that contain a hydrophilic (water-loving) and a hydrophobic (water-fearing) segment. One end of the molecule is thus polar in nature and dissolves in water, while the other end is nonpolar in nature, avoids water and dissolves in oil and other nonpolar compounds. When in water, surfactant molecules aim their polar ends at the water molecules, leaving the nonpolar ends sticking out like little electromagnets to attract nonpolar molecules. It is surmised that in a foamable composition such as those hereof, the polar ends may eject active oral care molecules like a rifle, and the non-polar ends lift tooth surface stain molecules like a chemical “lint roller” that loosens, breaks up and holds them onto the polar molecules, allowing them to be washed away with the water.

Either or both the first or second component in the two-component compositions hereof can also or alternatively include at least one surfactant in solid form. The ingredients in solid form, for example can include Pluronic F68, Sodium Carbonate anhydrous, Sodium Bicarbonate, potassium iodide and mixtures thereof. For a further example, surfactants hereof may include at least some difunctional block copolymer surfactant, such as those having terminal groups of primary hydroxyl groups, as well as those comprising a hydrophobic and a hydrophilic segment. Examples include Pluronic F68, Pluronic F88 and mixtures thereof.

The amount of a surfactant used herein can range, for example, from about 0.1% to about 5% by weight of the foamable composition, or for a further example, the surfactant can range from about 0.5% to about 3% by weight, and for a still further example, the surfactant can be less than about 1% by weight.

Faster acting foams may be found when a potassium iodide is added to one or the other of the first and second components, typically the second component, here the basic component. This will provide a quick acting foam with rapid radicals. However, a potassium iodide will form a colorful foam, usually yellowish or brownish, thus, a counteracting weak nitric acid may also be added to one or the other of the first and second components, usually to the first component, here the acidic component. Then, when mixed, the first and second components form a white foam which may have better cosmetic appeal, particularly in oral care and even more particularly in tooth whitening.

Perhaps surprisingly, the acidic compounds mentioned above, including tartaric, citric, nitric or phosphoric acid, if present, can also act as suitable stabilizers. The amount of acidic compound stabilizers, for example, ranges from about 0.1% to about 2% by weight of the composition.

For example, the time before 50% of the foam collapses is sufficiently long so that the teeth can be about 7 to about 8 shades lighter than their original tooth shade after three (3) applications of, for example, about 5 to about 10 minute duration when used in a photo-bleaching mode in a dental office setting. The advantage of the collapsibility of the foam formed by the composition of the present invention is that it collapses in a liquid of low viscosity, low surface tension and high ability to penetrate between teeth and between enamel rods, thus giving the bleaching composition the best opportunity to rapidly cause teeth whitening. On the other hand, the advantage of stable foams will insure that their adherence to teeth is better and thus will not running off or have to be constantly reapplied. Also the greater the volume of the foam, the higher the concentration of peroxide that can be used to cause rapid whitening of teeth without causing soft or hard tissue sensitivity issues. The ideal foam stability/collapsibility can also be easily adjusted according to customer desire.

For example, if present, any thickener will also preferably have a low viscosity so as not to inhibit the availability of active peroxides. The viscosity is generally, for example, less than about 5000 cps or perhaps less than about 1000 cps, or for a further example, less than about 500 cps, and further for example, less than about 100 cps and as fluid as less than 50 cps.

The composition of the present invention can also include other active ingredients, such as de-sensitizing agents and/or antimicrobial or anti-bacterial agents.

Even with improved efficiency and shorter treatment time, some patients may still experience sensitivity. Suitable desensitizing agents can include Eugenol and/or alkali nitrates such as potassium nitrate, sodium nitrate and lithium nitrate; and other potassium salts such as potassium chloride and potassium bicarbonate. A potassium nitrate and/or Eugenol may be the preferable agent used. The desensitizing agent may be disposed in either one or the other of the first and second components, typically in the first. The percent of desensitizing agent can be present up to about, for example, 5 percent by weight, further for example, up to about 4 percent by weight, and even further for example, up to about 3 percent by weight.

Antimicrobial or anti-bacterial agents may also be included, an example of which may be Eugenol. Eugenol may be included in either of the first and second components, typically in the first.

Further additives may include Calcium nitrate and/or Sodium mono and/or dibasic hydrate. These may be added to decrease surface tension and/or lower viscosity to provide a composition which is more “liquidy” and thus more able to get into the nooks and crannies. Stability may also be improved hereby. These additives may be added to either of the first and/or second components, typically the calcium nitrate into the first component and the sodium mono and/or dibasic hydrate into the second component. As mentioned above, potassium nitrate may alternatively and/or additionally be added for these surface tension and/or viscosity effects.

The composition of the present invention can also include other active ingredients, such as peroxide activators, re-mineralizing agents, and fluoridating agents.

The addition of peroxide activators can also increase the photobleaching efficiency of the foamable compositions of the present invention. Suitable peroxide activators include those with lower oxidative state transition metal salt. The metal salt may catalyze the bleaching action of the peroxide to produce faster effective bleaching at lower peroxide concentrations. The preferred transition metals are those of lower atomic numbers including lower atomic number transition metals such as those ranging from atomic number 21 to 30. Also, those with lower oxidative states may be more preferred, including, e.g., Iron(II), manganese(II), cobalt(II), copper(II) and mixtures thereof, and most preferably Iron(II), as in a ferrous gluconate. These may be added into either of the first and second components, typically in the second component. When used, only a very small amount of the transition metal salt is needed, for example, from about 0.01% by weight to about 4% by weight, further for example, from about 0.03% by weight to about 2% by weight, and even further for example, from about 0.04% to about 1% by weight. The peroxide activator can also include alkali salts such as potassium iodide, potassium chloride, sodium iodine, sodium chloride and combinations thereof.

Amorphous calcium compounds such as amorphous calcium phosphate (ACP), amorphous calcium phosphate fluoride (ACPF) and amorphous calcium carbonate phosphate (ACCP) amorphous calcium carbonate phosphate (ACCP), and amorphous calcium carbonate phosphate fluoride (ACCPF) can be used in remineralizing teeth. These amorphous compounds are disclosed in U.S. Pat. Nos. 5,037,639, 5,268,167, 5,437,857, 5,562,895, 6,000,341, and 6,056,930, the disclosure of each of which hereby being incorporated by reference in its entirety.

In addition to or as an alternative to amorphous calcium compounds, amorphous strontium compounds such as amorphous strontium phosphate (ASP), amorphous strontium phosphate fluoride (ASPF), amorphous strontium calcium phosphate (ASCP), amorphous strontium calcium carbonate phosphate (ASCCP), amorphous strontium carbonate phosphate fluoride (ASCPF) and amorphous strontium calcium carbonate phosphate fluoride (ASCCPF) may be included for use in re-mineralization, as noted above. Such compounds are disclosed in U.S. Pat. No. 5,534,244, the content of which hereby incorporated by reference in its entirety.

For example, the first component of the whitening system may include a source of phosphate and the second component may include a source of calcium or strontium. For example, the source of phosphate in the first component includes monosodium phosphate (NAH₂PO₄), disodium phosphate, tetrapotassium pyrophosphate and thereof. As discussed above, the second component, for example, may include a source of calcium or strontium, which, when the two gel components are mixed, combines with phosphate to form the various amorphous calcium and/or strontium phosphates. The source of phosphate may be, for example, present in an amount of from about 0.2% to about 5% by weight, further for example, between about 0.2% to about 4% by weight. The source of calcium, strontium or combinations thereof in the second component, for example, may include a calcium salt, a strontium salt, and thereof, further for example, a calcium salt such as calcium nitrate, in an amount of from about 0.25% by weight to about 1.5% by weight, for example, about 0.3% to about 1% by weight.

When the two components are mixed, the source of phosphate and the source of calcium, strontium or mixture can combine to form calcium phosphate. When applied to the teeth, the calcium phosphate can precipitate onto the surface of the teeth where it may be incorporated into hydroxyapatite, assisting in remineralization of the tooth enamel, as discussed in U.S. Pat. Nos. 5,037,639; 5,268,167; 5,460,803; 5,534,244; 5,562,895; 6,000,341; and 6,056,930 noted above.

In practice, it may in some embodiments be preferred to include as much phosphate as possible, as the phosphate salt further acts to adjust the pH of the first component. The pH of the system is from, for example, about 5 to about 8, further for example, from about 5.5 to about 6.5.

Note also, the fluoride-containing amorphous compounds described here may also be used in fluoridating teeth. Otherwise, as mentioned, fluorides may be added separately and then, many, if not all of the above amorphous compounds or solutions which form the amorphous compounds, when applied either onto or into dental tissue, particularly in the presence of fluoride, may operate to promote fluoridation. Such fluoridation or other mineralization may serve to assist in prevention and/or repair of dental weaknesses such as dental caries, exposed roots and dentin sensitivity.

In addition, optional additives including emulsifiers, flavorings, coloring agents, anti-plaque agents, anti-staining compounds, excipients such as emollients, preservatives, other types of stabilizers such as antioxidants, chelating agents, tonicity modifiers (e.g. sodium chloride, manitol, sorbitol or glucose), spreading agents, pH adjusting agents and water soluble lubricants, e.g. propylene glycol, glycerol or polyethylene glycol. The concentration of each may easily be determined by a person skilled in the art. Lecithin, a natural emulsifier found in soy and other plants, and gum arabic, which comes from the sap of certain species of acacia trees, can be added for use as an emulsifier, dispersant and/or wetting agent herein. Suitable preservatives may include benzalkonium chloride, parabens, chlorhexidine acetate, chlorhexidine gluconate, sorbic acid, potassium sorbitol, chlorbutanol and phenoxyethanol. Suitable emollients are those used for topical applications, as for example, di-n-octyl ether, fatty alcohol polyalkylene glycol ether, 2-ethylhexyl palmitate, and isopropyl fatty acid esters. An emollient, if used, may preferably be dispersed in the same part as the stabilizer, if used.

For increasing peroxide stability during storage, a 3% di-sodium EDTA may be added to the component containing the peroxide. Alternatively, stability may be enhanced by storing the product in a dark, cool, dry place or refrigerated. The acidic mixture of a peroxide in the first component containing the acid may also/alternatively stabilize or provide a tendency to stabilize the peroxide.

In packaging the oral care composition(s) hereof, any convenient means for effecting or maintaining the separation of the two components before use can be utilized. Note, this may include the alternative of encapsulating of the components in a unitary compartment, multi-phase environment. In an initial example, a single container can be compartmentalized so that the two components are housed in separate physical compartments and may be dispensed simultaneously and admixed prior to application on the teeth or otherwise in the oral cavity. The mixture may occur in or at the tip of a nozzle or a two-opening configuration may be provided where the components are not mixed until application. The containers may also include static mixers. Alternatively, the two components can be contained in separate containers from which the respective phases are dispensed for admixture just prior to use. Exemplary packaging alternatives are disclosed in U.S. Pat. Nos. 5,819,988, 6,065,645, 6,394,314, 6,564,972 and 6,698,622, the subject matter of which being incorporated herein by reference.

In one exemplary embodiment of the present invention, the two components are provided in separate chambers of a dual-barrel syringe. Immediately before use, the two components are mixed together in, for example in several embodiments, about a 1:2 to about a 5:1 ratio (first component to second component) by actuating the syringe, further for example, the fluids are mixed in the 1:1 ratio. The admixed oral care composition may be applied to the surface of the teeth or other oral tissue surface directly from the syringe. Other combinations of the components are contemplated by the present invention, depending on the percentage (%) variation of ingredients present in each component.

FIG. 1 shows a possible embodiment of a delivery system suited for dispensing the present invention. It is a dual-component syringe, preferably constructed of polypropylene and comprised of a plunger 1, dual-barrel body with integrated finger rest 2, and pistons 3. A mixing syringe tip comprised of a locking outer housing 4 and integrated static mixer 5 is attached. Two complimentary oral care compositions 6 and 7 can be contained within each isolated syringe barrel. When the user is ready to use the invention, the plunger is actuated towards the direction of the tip, resulting in the compositions becoming admixed within the syringe tip/static mixer component. The resultant homogenous foaming whitener 8 is expressed out of the dispenser and is ready for use.

FIG. 2 shows a possible embodiment of a delivery system suited for dispensing the present invention. It is a dual-component dispenser pump, preferably constructed of high density polypropylene and comprised of a plunger 9, upper housing with dual-barrel inner holding chambers 10, and fit into a lower housing 11 and base 12. Air-tight septum closures 13 keep the whitening composition within the holding chambers and are displaced upwards by a vacuum created when the plunger 9 is actuated. Two complimentary oral care compositions 16 and 17 can be contained within each of the isolated holding chambers 10. When the user is ready to use the invention, the plunger 9 is actuated downwards, resulting in the compositions becoming admixed within the locking mixing tip complex 14 comprising a static mixer component 15. The resultant homogenous foaming composition 18 is expressed out of the dispenser and is ready for use.

FIG. 3 shows a possible embodiment of a delivery system suited for dispensing the present invention. It is a dual-component dispenser pump, preferably constructed of high density polypropylene and comprised of a plunger 19, upper housing with dual-barrel inner holding chambers 20, and fit into a lower housing 21 and base 22. Air-tight septum closures 23 keep the whitening composition within the holding chambers and are displaced upwards by a vacuum created when the plunger 19 is actuated. Two complimentary oral care compositions 26 and 27 can be contained within each of the isolated holding chambers 20. When the user is ready to use the invention, the plunger 19 is actuated downwards, resulting in the compositions becoming expressed out of the dispenser from separate orifices. Two adjacent ribbons of the composition can be mixed together with a paint brush or swab, causing rapid foaming of the mixture. The foam can then be applied onto teeth. For storage of any remaining product, the pump has an integrated flip top closure 25 attached to the body with hinge 24.

In addition, any of the dispensers can also be fitted with a metering device for varying the proportion of each component in the final foam. The metering device can be adjusted to produce ratios of the two components from about 10:1 to about 1:10. The device can be in the form of a dispensing system which features a measuring mechanism that connects to two separate, interlockable bottles or like containers. By rotating the dispenser head, a precise mixing ratio of blended ingredients may be attained. In one specific embodiment, a dispenser head may include two pumps that offer varying proportions of volumetric dispensing that can be individually actuated in precise relationship to the positioning of an interior disc. This interior disc can be positioned precisely or locked into a specific ratio by rotating the dispenser head. The nozzle for metering dual dispenser pump bottle can either be a configuration in which both components are mixed with a static mixer incorporated within the tip thereof or a two-opening configuration where the components are not mixed until application. The use of metering devices can result in improved manufacturing efficiency, as fewer concentrations need to be made and the final concentration can be easily adjusted.

As noted above, the composition hereof may also be a multi-phase, foamable composition contained within a single unitary compartment, the composition including at least one acid compound in an aqueous phase and at least one basic compound in an oil phase. It is a well-known phenomenon that oil and water in general do not mix, and it is also a well-known phenomenon that they can be made to mix with the aid of foaming surfactants. Thus, upon mixing, the two-phases can combine to form a long-lasting, collapsible foam. When a preferred surfactant is used, the surfactant emulsifies and disperses the liquid by lowering the surface tension of both oil and water in order to effectively mix them together. Examples of solid surfactants are also difunctional block copolymer surfactants, similar to those listed above, including those having terminal groups of primary hydroxyl groups, as well as those comprising a hydrophobic and a hydrophilic segment. Examples include Pluronic F68, Pluronic F88 and mixtures thereof.

As discussed above, substantivity, i.e. the ability of a product to linger, is a desirable property in any whitening or other oral care composition. On the other hand when the desired property of a product is the ability to be rinsed off easily, a foaming surfactant would not be used. However, there is a desire in a whitening composition for both substantivity and the ability to be rinsed off easily. When this is needed, foams generated in a “foaming pump” can be used. Foaming pumps again can produce foams with all the desirable properties, while using a minimum of amount of surfactants, for example, of less than about 1.0%.

Alternatively and/or additionally, though not necessary, a foaming agent can also be a gaseous material. The gaseous material can be any inert gas or the gas can be generated by mixing a basic solution with an acid solution comprising a potassium salt such as potassium bromide or potassium chloride.

The metering dial of the pump dispenser may be used and may be set so that 50% of each component may be dispensed simultaneously and mixed through a nozzle containing a static mixer into a 20 ml plastic weighed boat. The plunger may be actuated about 6 times for dispensing about 5.0 milliliters into the weighed boat. Further mixing of both components may occur by swirling the mixture for about 5 to about 10 seconds with a small disposable dental nylon applicator brush or the like within the plastic well. The foam that may be produced may then be poured into a measuring cylinder. The volume of the expanded foam can then be measured and its collapse with time monitored.

The test results for this first example may be as follows. For the foam volume, collapse rate and pH; approximately four (4) ml of each component may be used and it may take approximately 10 seconds to actuate the dispenser about 10 times to achieve a desirable volume. A maximum volume of about 75 ml may then be obtained about 3 minutes after the last discharge. After about 7 minutes, the volume may decrease to about 35 ml, and after about 10 minutes the volume may decrease to about 20 ml. It may then take about 30 minutes before the foam collapses completely. The pH of the foam may be about 8.2. In another run, the amount of fluid used above may be about 3 ml from each chamber (about 6.0 ml total), and the total number of strokes of actuator may be about 6. The volume expansion may then go from about 6 ml to about 75 ml.

Thus described is a two-component foamable composition including a first component comprising at least one vinegar/acid compound; and a second component comprising at least one bicarbonate/sodium bicarbonate/alkaline source. Each of the components may be in an aqueous solution or otherwise in liquid form, though other phases are also usable herewith. Compositions hereof may also be multi-phase, foamable compositions with at least one acid in a liquid phase, usually aqueous and a carbonate in an oil phase. A clear xanthan gum may be a preferred foam stabilizer. Orange and/or lemon extracts or oils (or citric acid) may be used as well. Other embodiments may include at least one peroxide compound in a first component, typically the acidic component, often in an aqueous phase and at least one carbonate in a second component, the carbonate may be a foaming agent in such an example. Still further, other compositions may include a foamable one or two-component composition comprising at least one peroxide compound, wherein the resultant foam has a half life of from about 2 to about 60 minutes. Furthermore, a composition hereof may include a two-component foamable composition having a first component comprising at least one peroxide compound in an aqueous solution and at least one peroxide stabilizer in the form of an ion scavenger; and a second component comprising at least one foaming agent and at least one peroxide activator; wherein said activator promotes the rapid decomposition of the peroxide compound and additional foaming action not related to the foaming agent. A surfactant is also preferably included in one or the other or both components. Additionally, another alternative embodiment may include a light-activatable, two-component foamable composition having a first component comprising at least one peroxide compound; and a second component comprising at least one foaming agent and one lower oxidative state transition metal salt.

In one aspect, the foamable composition may be substantially free of gelling agents, thickeners, carbons, polymers or other ingredients that will tend to inhibit foaming. In yet a further aspect, the first component of the foamable composition may also include a de-sensitizing agent. In yet an additional aspect of the invention, the foamable composition may include at least one peroxide activator. In yet an additional aspect of the invention, the two-component foamable composition may contain peroxide compounds in both components. In yet an additional aspect of the invention, the foamable composition may include one or more foam stabilizers. In yet an additional aspect of the invention, the foamable composition may include at least one solvent suitable for solubilizing stains. In a further aspect, the first component of the foamable composition may include at least one source of calcium, strontium and mixtures thereof and the second component may include at least one source of phosphate.

In still yet another aspect of the invention, the two-component foamable composition may be provided in a double-barrel syringe. In still yet a further aspect of the invention, the two-component foamable composition may be provided in a double-barrel pumpable dispenser. The pumpable dispenser can be provided with a metering device for varying the proportion of each component in the final foam. The metering device may be adjusted to produce ratios of the two components of about 10:1 to 1:10.

In another aspect, the foamable composition may include ingredients capable of increasing the half life of the foam that is generated. The stabilizer may, for example, be added to facilitate the formation of a water-soluble, longer-lasting, collapsible foam structure.

Having described the invention with reference to accompanying illustrations and examples of the invention, it is contemplated that other changes can be made without departing from the spirit or scope of the invention as set forth in the appended claims.

1. A foaming oral care composition comprising two parts:
a vinegar part, and
a baking soda part.

2. An oral care composition according to claim 1 further comprising a clear xanthan gum for stabilizing the foam.

3. An oral care composition according to claim 3 wherein the clear xanthan gum has been hydrated for a period of hours.

4. An oral care composition according to claim 3 wherein the clear xanthan gum has been hydrated for a period of twenty four (24) hours.

5. An oral care composition according to claim 1 further comprising one or both of a natural lemon and a natural orange product.

6. An oral care composition according to claim 1 further comprising a whitening agent such as a peroxide or a salt of a chlorous acid.

7. An oral care composition according to claim 1 wherein each of the components may be in an aqueous solution or otherwise in liquid form.

8. An oral care composition according to claim 1 wherein the composition is disposed in a multi-phase, foamable form with at least one acid in a liquid aqueous phase and a carbonate in an oil phase.

9. (canceled)

10. An oral care composition according to claim 1 further comprising one or more of orange and/or lemon or other fruit extracts or oils, and citric acid.

11. (canceled)

12. An oral care composition according to claim 1 wherein the composition is substantially free of one or more of gelling agents, thickeners, carbons, polymers or other ingredients that will tend to inhibit foaming.

13-19. (canceled)

20. An oral care composition according to claim 1 disposed in a double-barrel syringe.

21. An oral care composition according to claim 1 wherein the composition is disposed in a double-barrel pumpable dispenser.

22. An oral care composition according to claim 1 wherein the composition is disposed in a double-barrel pumpable dispenser; and wherein the pumpable dispenser is provided with a metering device for varying the proportion of each component in the final foam.

23. An oral care composition according to claim 1 wherein the composition is disposed in a double-barrel pumpable dispenser; and wherein the pumpable dispenser is provided with a metering device for varying the proportion of each component in the final foam; and, wherein the metering device is adapted to be adjusted to produce ratios of the two components of about 10:1 to about 1:10.

24. An oral care composition according to claim 1 wherein the composition is disposed in a dispenser comprised of a sleeve with internal glass ampoules with a bristle applicator end.

25. A method of use of a foaming oral care composition of claim 1, the method comprising:
combining the vinegar part and the baking soda part to create a foaming composition;
applying the foaming composition to an oral feature to be cared for.

26. A method according to claim 25 wherein the operations of combining and applying occur in one or both of simultaneously and sequentially.

27. A method according to claim 25 wherein the operations of combining and applying occur sequentially wherein one of operations of combining and applying occurs after the other of the operations of combining and applying.

28. A method of use of a foaming oral care composition of two parts, a vinegar part, and a baking soda part; the method comprising:
mixing the two parts to obtain a foaming oral care composition;
applying the oral care composition into an oral cavity.

29. A method of making a foaming oral care composition of two parts;
namely, a vinegar part, and a baking soda part, the method comprising:
preparing a vinegar part, and preparing a baking soda part; and,
maintaining the vinegar and baking soda parts separate until use.

30. A method according to claim 29 further including an operation for combining the vinegar and baking soda parts to create an oral care foam.

31. A method of making according to claim 29 further including one or more of adding one of a lemon oil or citric acid, or an orange oil or a stabilizer to one or the other or both of the vinegar and baking soda parts.

32. A method according to claim 29 further including the addition of an oxidizing whitener to one of the vinegar and baking soda parts.

33. A method of use according to claim 28 in which the oral care composition is used in a multi-component system including one or more of a two-, three- or four-component system, wherein the multi-component system includes one or more of an enhancer composition, one or more foaming components and a finishing composition.
